(19) United States
(12) Patent Application Publication
(10) Pub. No.: US 2014/0228967 A1
(43) Pub. Date: Aug. 14, 2014

(54) BIOCORRODIBLE IMPLANT WITH ANTI-CORROSION COATING

(71) Applicant: BIOTRONIK AG, Buelach (CH)

(72) Inventor: Eric Wittchow, Gunzenhausen (DE)

(73) Assignee: BIOTRONIK AG, Buelach (CH)

(21) Appl. No.: 14/166,206

(22) Filed: Jan. 28, 2014

Related U.S. Application Data
(60) Provisional application No. 61/763,956, filed on Feb. 13, 2013.

(51) Int. Cl. A61L 31/14; A61F 2/82; A61L 31/16

(52) U.S. Cl. CPC A61L 31/148; A61L 31/16; A61F 2/82
USPC 623/23.7

(57) ABSTRACT

An implant, preferably a stent, which has a main body made of magnesium or a biocorrodible magnesium alloy and a corrosion-inhibiting passivation layer covering the main body. The passivation layer is characterized in that it contains a composite of a biodegradable polymer and nanoparticles of clay minerals.

### CROSS REFERENCE TO RELATED APPLICATIONS

This application claims benefit of priority to U.S. provisional patent applications Ser. No. 61/763,965 filed Feb. 13, 2013; the contents of which are herein incorporated by reference in their entirety.

### TECHNICAL FIELD

The invention relates to a biocorrodible implant based on magnesium or a biocorrodible magnesium alloy, of which the surface has an anti-corrosion coating.

### BACKGROUND

An implant is generally understood to mean any medical device formed from one or more materials, which is inserted intentionally into the body and is covered either in part or completely by an epithelial surface. Implants can be sub-divided in terms of the period of use into temporary and permanent implants. Temporary implants remain in the body for a limited period of time. Permanent implants are intended to remain permanently in the body. In the case of implants, a further distinction can be made between prostheses and artificial organs. A prosthesis is a medical device that replaces limbs, organs or tissues of the body, whereas an artificial organ is understood to be a medical device that replaces the function of a bodily organ, either in part or completely. The above definitions include, for example, implants such as orthopedic or osteosynthetic implants, cardiac pacemakers and defibrillators, and vascular implants.

In particular, the implantation of stents has become established as one of the most effective therapeutic measures in the treatment of vascular diseases. Stents are used to perform a supporting function in a patient's hollow organs. For this purpose, stents of conventional design have a filigree supporting structure formed from metal struts, which is initially pro-vided in a compressed form for insertion into the body and is expanded at the site of application. One of the main fields of application of such stents is the permanent or temporary widening and maintained opening of vascular constrictions, in particular of constrictions (stenoses) of the coronary vessels. In addition, aneurysm stents are known for example, which are used primarily to seal the aneurysm.

Stents have a peripheral wall of sufficient supporting strength to hold open the constricted vessel to the desired extent and also a tubular main body, through which the flow of blood continues unimpeded. The peripheral wall is generally formed by a mesh-like supporting structure, which allows the stent to be introduced in a compressed state with small outer diameter as far as the narrowed point to be treated of the respective vessel, where the stent can then be expanded with the aid of a balloon catheter for example until the vessel has the desired, increased inner diameter. Alternatively, shape-memory materials such as Nitinol have the ability to self-expand if there is no restoring force that holds the implant at a small diameter. The restoring force is generally exerted onto the material by means of a protective tube.

The implant, in particular the stent, has a main body formed from an implant material. An implant material is a non-living material, which is used for an application in the field of medicine and interacts with biological systems. Basic preconditions for the use of a material as implant material that comes into contact with the bodily environment when used as intended is its compatibility with the body (biocompatibility). Biocompatibility is understood to mean the ability of a material to induce a suitable tissue response in a specific application. This includes an adaptation of the chemical, physical, biological and morphological surface properties of an implant to the receiver tissue with the objective of a clinically desired interaction. The biocompatibility of the implant material is also dependent on the progression over time of the response of the biosystem into which the material has been implanted. Relatively short-term irritation and inflammation thus occur and may lead to tissue changes. Biological systems therefore respond differently according to the properties of the implant material. The implant materials can be divided into bioactive, bioinert and degradable/resorbable materials in accordance with the response of the biosystem.

Implant materials comprise polymers, metal materials and ceramic materials (for example as a coating). Biocompatible metals and metal alloys for permanent implants include stainless steels for example (such as 316L), cobalt-based alloys (such as CoCrMo cast alloys, CoCrMo forged alloys, CoCrWNi forged alloys and CoCrNiMo forged alloys), pure titanium and titanium alloys (for example cp titanium, TiAl6V4 or TiAl6Nb7) and gold alloys. In the field of biocorrodible implants, in particular stents, the use of magnesium or pure iron as well as biocorrodible master alloys of the elements magnesium, iron, zinc, molybdenum and tungsten is recommended.

For the purposes of the present invention, merely metal implant materials that consist completely or in part of magnesium or biocorrodible magnesium alloys (magnesium-containing alloys) are of interest.

A limitation with the use of biocorrodible magnesium alloys is the rapid degradation of the material in a physiological environment. Both the fundamental principles of magnesium corrosion and a large number of technical methods for improving the corrosion behavior (in the sense of boosting the protection against corrosion) are known from the prior art. For example, it is known that the addition of yttrium and/or further rare earth metals of a magnesium alloy provides slightly increased resistance to corrosion in seawater.

A starting point for improving corrosion behavior lies in producing an anti-corrosion layer on the shaped article consisting of magnesium or a magnesium alloy. Known methods for producing an anti-corrosion layer have been previously developed and optimized from the viewpoint of a technical use of the shaped article, but not from the viewpoint of a medical use in biocorrodible implants in a physiological environment. These known methods comprise: the application of polymers or inorganic cover layers, the production of an enamel, the chemical conversion of the surface, hot-gas oxidation, anodizing, plasma spraying, laser beam remelting, PVD methods, ion implantation or coating.

Conventional technical fields of use of shaped articles made of magnesium alloys outside the field of medicine generally require extensive prevention of corrosive processes. The objective of most technical methods is accordingly complete elimination of corrosive processes. By contrast, the objective for improving the corrosive behavior of biocorrodible magnesium alloys should not lie in complete prevention, but merely in the suppression of corrosive processes. For this reason alone, most known methods for the production of anti-corrosion layers on magnesium are unsuitable. Furthermore, toxicological aspects also have to be taken into account for medical use. Corrosive processes are also highly dependent on the medium in which they take place, and it may not therefore be possible to transfer, without limitation, the anti-corrosion findings obtained under conventional environmental conditions in the technical field to the processes in a physiological environment. Lastly, with a large number of medical implants the mechanisms forming the basis of corrosion may also differ from conventional technical applications of the material. For example, stents, surgical material or clips are mechanically deformed during use, and therefore the sub-process of stress corrosion cracking could be of considerable importance during the breakdown of these shaped articles.

The main body of some implants, such as stents in particular, is subject locally during use to plastic deformation of varying strength. Conventional methods for inhibiting corrosion, such as the generation of a dense magnesium oxide cover layer, do not lead to the desired result in this instance. The ceramic properties of such a cover layer would lead to local flaking or at least cracking of the cover layer. The corrosion would therefore occur in an uncontrolled manner and in particular there would be a risk that the corrosion is accelerated in the regions of the implant subject to considerable mechanical stress.

The application of non-degradable, polymer passivation layers can indeed inhibit the breakdown of the implant, but this contradicts the fundamental idea of a fully degradable implant, since the polymer material of the passivation layer remains in the body. A promising alternative is therefore the use of biodegradable polymers as material for a passivation layer for implants based on biodegradable magnesium materials.

It is known from US 2009-0240323 A1, US 2010-0076544 A1 and US 2011/0076319 A1 to coat a magnesium stent with a biodegradable polymer coating as a passivation layer. The breakdown of the main body made of magnesium or a magnesium alloy should therefore be decelerated. For example, polylactides, polyhydroxyalkanoates, polycaprolactones, aliphatic polyesters, aromatic copolyesters and polyester amides are possible biodegradable polymer coating materials. In spite of these promising approaches, it has not yet been possible to satisfactorily delay to a sufficient degree the degradation of the metal main body of the stent.

### SUMMARY OF THE INVENTION

One or more of the discussed problems of the prior art can be overcome or at least mitigated with the aid of the implant according to the invention. The implant, preferably a stent, has a main body made of magnesium or a biocorrodible magnesium alloy and a corrosion-inhibiting passivation layer covering the main body. The passivation layer is characterized in that it contains a composite of a biodegradable polymer and nanoparticles of clay minerals.

### DESCRIPTION OF PREFERRED EMBODIMENTS

The invention is based on the finding that the water transmission rate of the passivation layer can be reduced quite considerably with the aid of a composite of biodegradable polymers and nanoparticles of clay minerals. It has been demonstrated in some tests that even a low water transmission rate is a key factor for sufficient function of the passivation layer. The degradation protection thus depends substantially on the water transmission rate, which is determined by the diffusion coefficients and layer thickness. By admixing the clay minerals, the water transmission rate of the passivation layer should be lowered in particular below a value of the standardized water vapor transmission rate (WVTR) of 50 g/m² per day.

In the present case, the term “clay minerals” is understood to mean minerals that are pre-dominantly present in the finest grain size (grain size≦2 μm) and are layered silicates. Clay minerals consist of two characteristic components: a tetrahedral layer (corner-linked Slat tetrahedron, Si substituted by Al in part) and an octahedral layer (edge-linked AlO₆ octahedron, Al substituted by Mg in part).

Depending on the arrangement of these layers a distinction is made between 1:1 clay minerals (two-layer clay minerals, for example kaolinite or chrysotile), 2:1 clay minerals (three-layer clay minerals, for example illite) and 2:1:1 clay minerals (four-layer clay minerals, for example chlorite). A negative layer charge is produced by the substitution (pre-dominantly of quadrivalent Si by trivalent Al in the tetrahedral layer of or trivalent Al by bivalent Mg in the octahedral layer) and is neutralized by the inclusion of cations in the intermediate layer. The layer charge of the 1:1 clay minerals is always zero. The 2:1 clay minerals are further divided in accordance with their layer charge into the talc-pyrophyllite group, smectite group (for example montmorillonite, beidellite, nontronite, saponite or hectorite), vermiculite-illite group and mica group. Clay minerals with non-integer layer charges have the capacity to swell, that is to say for temporary and reversible water uptake in its intermediate layers. Alternatively, the layer charge in the octahedral layer can also be compensated if only two of three octahedrons are occupied (dioctahedral clay minerals, such as kaolinite; trioctahedral clay minerals, for example chrysotile). The clay mineral is preferably montmorillonite or saponite.

For use as a filler in the passivation layer formed of the biodegradable polymer, the clay minerals are preferably modified beforehand so as to make them lipophilic. They thus lose their high capacity for water uptake, but can be mixed with the biodegradable polymers. The modification can take place by replacing inorganic ions in the mineral with substituted ammonium ions or phosphonium ions (organo-modified clay minerals; see Perrine Bordes, Eric Pallet, Luc Averous, Nanobiocomposites: Biodegradable polyester/nanoclay systems, Progress in Polymer Science 34 (2009), 125-155). The organo-modified clay mineral is preferably montmorillonite or saponite. The clay minerals used as a filler generally have layer thicknesses of approximately 1 nm and layer lengths of up to 2 μm.

The compound of polymer matrix and clay mineral can be achieved in three ways:
a) Solvent Intercalation Route, in which the mineral is bloated in a polymer-containing solution, whereby the macromolecule diffuses into the cavities in the clay earth;
b) In-situ Intercalation Method, in which the mineral is bloated in a monomer-containing solution before it is polymerized; or
c) Melt Intercalation Process, in which the polymer is mixed in the molten state, for example during extrusion.

It is also preferred if the biodegradable polymer is selected from the group of polylactides, polyhydroxyalkanoates, polycaprolactones, aliphatic polyesters, aromatic copolyesters and polyester amides. The biodegradable polymer polyhydroxyalkanoate is particularly preferred. The aforementioned biodegradable polymers can be converted particularly easily into the desired composites and generally have the degradation behavior suitable for the purposes according to the invention.

The passivation layer may contain further additives and preferably also an active ingredient, which are released after implantation.

The pharmacological active ingredient is preferably selected from the group of limus compounds, preferably sirolimus (rapamycin), and cytostatics, preferably paclitaxel.

In the present case, “magnesium alloy” is understood to mean a metal structure of which the main component is magnesium. The main component is the alloy component of which the proportion by weight in the alloy is greatest. A proportion of the main component is preferably more than 50% by weight, in particular more than 70% by weight. The composition of the alloy is to be selected such that said alloy is biocorrodible. Artificial plasma, as stipulated in accordance with EN ISO 10993-15:2000 for biocorrosion tests (composition NaCl 6.8 g/l, CaCl₂ 0.2 g/l, KCl 0.4 g/l, MgSO₄ 0.1 g/l, NaHCO₃ 2.2 g/l, Na₂HPO₄ 0.126 g/l, NaH₂PO₄ 0.026 g/l), is used as a test medium for testing the corrosion behavior of a possible alloy. A sample of the alloy to be tested is stored for this purpose in a closed sample container with a defined amount of the test medium at 37° C. and pH 7.38. The samples are removed at intervals over time (adapted to the expected corrosion behavior) from a few hours to several months and are examined in a known manner for signs of corrosion. The artificial plasma according to EN ISO 10993-15:2000 corresponds to a blood-like medium and therefore constitutes one possibility for reproducibly reconstructing a physiological environment within the meaning of the invention.

Implants within the meaning of the invention are devices introduced into the body via a surgical procedure and include fixing elements for bones, for example screws, plates or nails, surgical suture material, intestinal clamps, vessel clips, prostheses in the region of hard and soft tissue, and anchoring elements for electrodes, in particular of pacemakers or defibrillators. The implant consists completely or in part of the biocorrodible material. The implant is preferably a stent.

### Exemplary Embodiment

The nanocomposite is produced by the solution intercalation method. Organo-modified montmorillonite (OMMT) is added in the form of powder to a solution of PHBV (poly(3-hydroxybutyrat-co-3-hydroxyvalerate)) in chloroform in order to obtain a proportion by weight of 5% by weight of OMMT (production occurs similarly to Chen et al., Journal of Material Science Letter 21 (2002), 1587-1589). The dispersion is dispersed in an ultra-sonic bath for a further three hours and is heated under reflux for 1 h at 60° C. before further processing. Once cooled to room temperature, the solution is applied to the surface of a stent by spraying, said stent being formed from a biocorrodible magnesium alloy.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

What is claimed is:

1. An implant comprising a main body made of magnesium or a biocorrodible magnesium alloy and a corrosion-inhibiting passivation layer covering the main body, wherein the passivation layer contains a composite of a biodegradable polymer and nanoparticles of clay minerals.

2. The implant as claimed in claim 1, wherein the clay mineral is an organo-modified

3. The implant as claimed in claim 1, wherein the clay mineral is montmorillonite or saponite.

4. The implant as claimed in claim 1, wherein the biodegradable polymer is selected from the group consisting of a polylactide, a polyhydroxyalkanoate, a polycaprolactone, an aliphatic polyester, an aromatic copolyester and a polyester amide.

5. The implant as claimed in claim 4, wherein the biodegradable polymer is the poly-hydroxyalkanoate.

6. The implant as claimed in claim 1, wherein the implant is a stent.

7. The implant as claimed in claim 1, wherein the passivation layer contains additives and/or an active ingredient, optionally a pharmacological active ingredient.

8. The implant as claimed in claim 7, wherein the pharmacological active ingredient is a limus compound, optionally sirolimus (rapamycin), or a cytostatic, optionally paclitaxel.
